(19) United States
(12) Patent Application Publication
(10) Pub. No.: US 2002/0058026 A1
(43) Pub. Date: May 16, 2002

(54) HMG CoA reductase inhibitor medications combined wih CoEnzyme Q-10

(76) Inventor: Milton Hammerly, Parker, CO (US)

Correspondence Address: Christopher J. Whewell, Western Patent Group, 6020 Tonkowa Trail, Georgetown, TX, 78628, US

(21) Appl. No.: 09/991,510

(22) Filed: Nov. 10, 2001

Related U.S. Application Data
(60) Provisional application No. 60/246,995, filed on Nov. 13, 2000.

(51) Int. Cl. A61K 38/43; A61K 31/225; A61K 31/401

(52) U.S. Cl. 424/94.1; 514/423; 514/547

(57) ABSTRACT

Provided herein are medicinal compositions of matter that comprise an HMG CoA reductase inhibitor medication component and a CoEnzyme Q-10 component. Through use of the compositions of the invention a reduction of potential adverse physiologic effects is observed in the treatment of hypercholesterolemia, mixed dyslipidemia, and coronary artery disease. The invention also provides procedures for administering the composition to a patient who is afflicted with hypercholesterolemia, mixed dyslipidemia, or coronary artery disease.

This application claims the benefit of U.S. Provisional Application No. 60/246,995 filed Nov. 13, 2000 the entire contents of which are herein incorporated by reference.

### TECHNICAL FIELD

The present invention relates to compositions of matter useful in the prevention and treatment of diseases such as atherosclerotic cardiovascular disease (ASCVD). More particularly, the present invention encompasses a method for treatment and prevention of ASCVD including the use of a medicinal compositions of matter comprising an HMG CoA reductase inhibitor and CoEnzyme Q-10. The compositions according to the invention are suitable for oral administration, or administration by any conventional means.

### BACKGROUND

Atherosclerotic cardiovascular disease (ASCVD) is the leading cause of death in most industrial countries. This disease involves large, medium and small arteries throughout the body. In addition to family history, the atherogenic risk factors are known to include smoking, hypertension, diabetes mellitus, cholesterol abnormalities and homocysteinuria. The presence of each additional risk factor markedly aggravates the potential for development of the disease. Although seemingly diverse, the risk factors all damage the artery wall and effect formation of thrombosis.

In the aorta, the largest artery, the artery wall damage may lead to aortic aneurysm or embolism. ASCVD in medium and small arteries can result in sudden occlusion of the vessel or progressive narrowing of the arterial lumen. The symptoms of persons with this disease are dictated by the organs supplied by the effected arteries. Lumenal narrowing of the arteries supplying the heart with blood is called coronary artery disease (CAD). The symptoms include angina, unstable angina, myocardial infarction (MI) and sudden death. Cerebral vascular disease (CVD) symptoms include progressive neural deterioration, transient ischemic attack (TIA), seizures, and cerebral vascular accident (CVA), i.e., stroke. Kidney effects include hypertension, renal infarction and renal failure. Abdominal vascular insufficiency results in abdominal angina and bowel infarction. Peripheral vascular disease (PVD) symptoms include intermittent claudication, gangrene and amputation.

Currently accepted clinical treatment of ASCVD includes prescription medications such as beta blockers, angiotensin converting enzyme (ACE) inhibitors, calcium channel blockers, and cholesterol lowering medication. In addition, aspirin is prescribed by cardiologists in many ASCVD conditions. The affect of arterial thrombus formation contributes to vascular wall damage and acts as the terminal event in a condition like MI. Thrombus formation is a complex interaction of platelets, coagulation factors, and damage to the intima and endothelial layer of the artery wall. Certain risk factors and thrombosis on the vascular wall contribute to damage the inner vascular wall and expose vascular endothelium. Circulating platelets adhere to the exposed subendothelium collagen. The Von Willebrand factor binds platelets causing adhesion. This initiates a series of progressive events stimulating platelet aggregation, and includes prostaglandin H2 and thromboxane A2. Circulating fibrinogen converts to fibrin with the formation of fibrin strands which secure the platelet mass to the arterial wall. If the reaction ceases at this juncture, the artery wall has been weakened and can deteriorate again at any time, and the lumen of the vessel has been diminished to some degree. If the event is self propagating the vessel can occlude and result in infarction of the affected organ.

Hypercholesterolemia and mixed dyslipidemia, are also known risk factors for coronary artery disease. The class of medications known as HMG CoA reductase inhibitors have been shown to be efficacious in lowering serum cholesterol levels and reducing both morbidity and mortality from coronary heart disease. The mechanism of action of the HMG CoA reductase inhibitors is believed to be the inhibition of the activity of a key enzyme (HMG CoA reductase) needed in the hepatic biosynthesis process of producing cholesterol.

A well-documented side effect of blocking the activity of HMG CoA reductase is that levels of CoEnzyme Q-10 (a.k.a. ubiquinone) decline. However, the therapeutic, cholesterol-lowering effect of the HMG CoA reductase inhibiting medications is not in any way dependent on low levels of CoEnzyme Q-10. In other words, achieving low levels of CoEnxyme Q-10 is not necessary for such medications to treat hypercholesterolemia or mixed dyslipidemia. Further, low levels of CoEnzyme Q-10 have been associated with heart failure, hypertension, and certain types of cancer. Research has documented therapeutic benefits of CoEnzyme Q-10 supplementation in patients with low levels of CoEnzyme Q-10.

Available HMG CoA reductase inhibiting medications such as BAYCOL® (cerivastatin), LESCOL® (fluvastatin), LIPITOR® (atorvastatin), MEVACOR® (lovastatin), PRAVACHOL® (pravastatin), ZOCOR® (simvastatin) all reduce serum cholesterol (desirable) as well as CoEnzyme Q-10 levels (undesirable). Since many patients being treated for hypercholesterolemia or mixed dyslipidemia are also at risk for heart failure and hypertension, it would be physiologically and clinically advantageous to prevent the depletion of CoEnzyme Q-10 levels caused by treatment with HMG CoA reductase inhibitors in order to decrease the risk of cardiovascular co-morbidities.

### SUMMARY OF THE INVENTION

The present invention is concerned with medicinal compositions that reduce potential negative physiologic side effects of cholesterol lowering medications. Compositions according to the invention are comprised of an HMG CoA reductase inhibitor medication component and CoEnzyme Q-10 component. The invention is also concerned with a process for treating a human patient who is afflicted with hypercholesterolemia, mixed dyslipidemia, or coronary artery disease which comprises the steps of providing a composition according to the invention and introducing such a composition into the patient's body.

### DETAILED DESCRIPTION

The present invention is directed at combinations for administration to humans which combinations comprise an HMG CoA reductase inhibitor medicament in combination with Co-Enzyme Q-10. HMG CoA reductase inhibitors such as BAYCOL® (cerivastatin), LESCOL® (fluvastatin), LIPITOR® (atorvastatin), MEVACOR® (lovastatin), PRAVACHOL® (pravastatin), ZOCOR® (simvastatin) in general desirably reduce serum cholesterol. However, an unfortunate side effect is that they also simultaneously reduce serum CoEnzyme Q-10 levels as well.

According to the present invention, the reduction in CoEnzyme Q-10 levels normally associated with the administration of an HMG CoA reductase inhibitor are alleviated or eliminated. This is made possible by the inclusion of Co-Enzyme Q-10 in a medicament which contains an HMG CoA reductase inhibitor. Thus, the present invention includes providing CoEnzyme Q-10 to HMG CoA reductase inhibitors simultaneously with HMG CoA reductase inhibitors. Such combinations reduce, and in some cases may eliminate the negative physiological consequences of therapy with HMG CoA reductase inhibitors. The use of such novel combination to treat hypercholesterolemia, mixed dyslipidemia or coronary artery disease prevents many adverse effects, and enhances the treatment of co-morbid cardiovascular conditions.

The present invention embraces therapeutically effective compositions of matter which contain any HMG CoA reductase inhibitor and CoEnzyme Q-10 in combination with each other. In one preferred embodiment, a combination according to the invention may be introduced into the human body via oral administration by the patient themselves. In other embodiments, a combination according to the invention may be administered enterally or parenterally, as such methods of administration are well-known in the art.

### HMG CoA Reductase

HMG Co-A Reductase Inhibitors are compounds termed such since in general they inhibit 3-hydroxy-3-methylglutaryl coenzyme A reductase. This enzyme catalyzes the conversion of HMG Co-A to mevalonate, which is an early and rate-limiting step in the biosynthesis of cholesterol. Known HMG Co-A reductase inhibitors include, for example, mevastatin (disclosed in U.S. Pat. No. 3,883,140), lovastatin, also referred to as mevinolin (disclosed in U.S. Pat. No. 4,231,938), pravastatin (disclosed in U.S. Pat. No. 4,346,227), simvastatin also referred to as synvinolin (disclosed in U.S. Pat. Nos. 4,444,784 and 4,450,171), fluvastatin (disclosed in U.S. Pat. No. 4,739,073) and atorvastatin (disclosed in U.S. Pat. No. 5,273,995) mevastatin, fluindostatin, cerivastatin, and compactin, among others. For purposes of the present invention, “HMG CoA reductase inhibitor” means any substance known to be capable of inhibiting the operation of 3-hydroxy-3-methylglutaryl coenzyme A reductase.

With regards to the amounts of HMG CoA reductase inhibitor medications present in a combination according to the invention, the amount of each HMG CoA reductase inhibitor present varies upon the effective dosage of each particular HMG CoA reductase inhibitor in each case. For the HMG CoA reductase inhibitor BAYCOL® (cerivastatin), the dosage range is about 0.2 to 0.8 milligrams daily; for LESCOL® (fluvastatin), the dosage range is about 20 to 80 milligrams daily; for LIPITOR® (atorvastatin), the dosage range is about 10 to 80 milligrams daily; for MEVACOR® (lovastatin), the dosage range is about 20 to 80 milligrams daily; for PRAVACHOL® (pravastatin) the dosage range is about 10 to 40 milligrams daily; and for ZOCOR® (simvastatin), the dosage range is about 5 to 80 milligrams daily. All of the above milligram quantities are administered on a weight basis relative to the weight of the patient, as is the case for most medicaments, as is well-known in the art.

### Co-Enzyme Q-10

The nutrient Coenzyme Q-10 is found in every cell in the body, thus its other name, ubiquinone (“ubiquitous quinone”). Ubiquinone is a naturally-occurring substance with a molecular structure that is similar to vitamin K. Because the body must have energy available to perform even the simplest operation, Coenzyme Q-10 is considered essential for the body's cells, tissues and organs. Even though the body has the ability to produce Coenzyme Q-10, deficiencies have been reported in a range of clinical conditions. Supplementation of the coenzyme may thus help the body to guard against a possible deficiency. Aging is considered one reason for a deficiency, since the liver loses its ability to synthesize Coenzyme Q-10 as one gets older. Besides aging, poor eating habits, stress, and infection affect the body's ability to provide adequate amounts of Coenzyme Q-10. Coenzyme Q-10 also improves the efficiency of energy production at the cellular level. It has also demonstrated excellent results in clinical trials on periodontal disease by speeding up healing time, reducing gum pockets, and improving other factors associated with gum disease.

The optimum Co-Enzyme Q-10 dosage levels or amount present in a combination according to the invention are dependent on the specific dose and type of HMG CoA reductase inhibitor medications used. The optimum level of CoEnzyme Q-10 in a combination according to the invention is any amount in the range of between about 5 milligrams to about 150 milligrams, including every milligram therebetween, based upon a patient having body mass of about 70 kilograms.

Consideration must be given to the fact that although this invention has been described and disclosed in relation to certain preferred embodiments, obvious equivalent modifications and alterations thereof will become apparent to one of ordinary skill in this art upon reading and understanding this specification and the claims appended hereto. Accordingly, the presently disclosed invention is intended to cover all such modifications and alterations, and is limited only by the scope of the claims which follow.

I claim:

1) A medicinal composition useful in the treatment of hypercholesterolemia, mixed dyslipidemia and coronary artery disease comprising:
a) an HMG CoA reductase inhibitor component; and
b) a CoEnzyme Q-10 component.

2) A composition as set forth in claim 1 wherein said HMG CoA reductase inhibitor medication is selected from the group consisting of: BAYCOL® (cerivastatin), LESCOL® (fluvastatin), LIPITOR® (atorvastatin), MEVACOR® (lovastatin), PRAVACHOL® (pravastatin), ZOCOR® (simvastatin), and derivatives of the foregoing.

3) A composition as set forth in claim 1 wherein said CoEnzyme Q-10 is present in any of its known forms.

4) A composition according to any of claim 1 wherein said HMG CoA reductase inhibitor component is present in any amount between 0.05% and 99.5% by weight based upon total weight of the composition, including every hundredth percentage therebetween.

5) A composition according to any of claim 1 wherein said CoEnzyme Q-10 component is present in any amount between 0.05% and 99.5% by weight based upon total weight of the composition, including every hundredth percentage therebetween.

6) A single-dose medicament useful for treating hypercholesterolemia, mixed dyslipidemia and coronary artery disease comprising a composition according claim 1, wherein said HMG CoA reductase inhibitor is BAYCOL® (cerivastatin) and wherein said HMG CoA reductase inhibitor is present in a quantity of between 0.05 to 0.8 milligrams, including every milligram therebetween, and wherein the quantity of CoEnzyme Q-10 is at least 2 milligrams.

7) A single-dose medicament useful for treating hypercholesterolemia, mixed dyslipidemia and coronary artery disease comprising a composition according claim 1, wherein said HMG CoA reductase inhibitor is LESCOL® (fluvastatin) and wherein said HMG CoA reductase inhibitor is present in a quantity of between 5 to 80 milligrams, including every milligram therebetween, and wherein the quantity of CoEnzyme Q-10 is at least 2 milligrams.

8) A single-dose medicament useful for treating hypercholesterolemia, mixed dyslipidemia and coronary artery disease comprising a composition according claim 1, wherein said HMG CoA reductase inhibitor is LIPITOR® (atorvastatin) and wherein said HMG CoA reductase inhibitor is present in a quantity of between 3 to 80 milligrams, including every milligram therebetween, and wherein the quantity of CoEnzyme Q-10 is at least 2 milligrams.

9) A single-dose medicament useful for treating hypercholesterolemia, mixed dyslipidemia and coronary artery disease comprising a composition according claim 1, wherein said HMG CoA reductase inhibitor is MEVACOR® (lovastatin) and wherein said HMG CoA reductase inhibitor is present in a quantity of between 5 and 80 milligrams, including every milligram therebetween, and wherein the quantity of CoEnzyme Q-10 is at least 2 milligrams.

10) A single-dose medicament useful for treating hypercholesterolemia, mixed dyslipidemia and coronary artery disease comprising a composition according claim 1, wherein said HMG CoA reductase inhibitor is PRAVACHOL® (pravastatin) and wherein said HMG CoA reductase inhibitor is present in a quantity of between 3 and 40 milligrams, including every milligram therebetween, and wherein the quantity of CoEnzyme Q-10 is at least 2 milligrams.

11) A single-dose medicament useful for treating hypercholesterolemia, mixed dyslipidemia and coronary artery disease comprising a composition according claim 1, wherein said HMG CoA reductase inhibitor is ZOCOR® (simvastatin) and wherein said HMG CoA reductase inhibitor is present in a quantity of between 1 and 80 milligrams, including every milligram therebetween, and wherein the quantity of CoEnzyme Q-10 is at least 2 milligrams.

12) A composition useful for treating hypercholesterolemia, mixed dyslipidemia and coronary artery disease comprising a composition according claim 1, wherein said HMG CoA reductase inhibitor is selected from the group consisting: of BAYCOL® (cerivastatin), LESCOL® (fluvastatin), LIPITOR® (atorvastatin), MEVACOR® (lovastatin), PRAVACHOL® (pravastatin), ZOCOR® (simvastatin), and derivatives of the foregoing, and wherein said HMG CoA reductase inhibitor is present in any quantity of between 1 milligrams and 100 milligrams, including every milligram therebetween, and wherein the quantity of CoEnzyme Q-10 is present in any quantity of between 1 milligrams and 100 milligrams, including every milligram therebetween.

13) A method of treating a patient showing symptoms of hypercholesterolemia, mixed dyslipidemia and coronary artery disease, the method comprising:
administration to said patient of a therapeutically effective amount of an HMG
CoA reductase inhibitor component in synergistic combination with a
therapeutically effective amount of a Co-Enzyme Q-10 component, wherein the HMG CoA reductase inhibitor component is administered to alleviate symptoms of hypercholesterolemia, mixed dyslipidemia, or coronary artery disease.

14) A method according to claim 13 wherein the administration is oral.

15) A method according to claim 13 wherein the administration is parenteral.

16) A method according to claim 13, wherein the HMG CoA reductase inhibitor component is selected from a group consisting of: BAYCOL® (cerivastatin), LESCOL® (fluvastatin), LIPITOR® (atorvastatin), MEVACOR® (lovastatin), PRAVACHOL® (pravastatin), ZOCOR® (simvastatin), and derivatives of the foregoing.

17) A method according to claim 13, wherein the HMG CoA reductase inhibitor component is compounded into a single oral unit with the Co-Enzyme Q-10.

18) A method according to claim 17, wherein the single oral unit is a tablet.

19) A method according to claim 17, wherein the single oral unit is a capsule.

20) A method according to claim 13, wherein the HMG CoA reductase inhibitor medication is dissolved or suspended in an oil.

21) A method according to claim 20 wherein the dissolved or suspended HMG CoA reductase inhibitor medication is placed in a single oral softgelatin capsule.

22) A method according to claim 13, wherein the HMG CoA reductase inhibitor component is formed into a first single oral unit, and the Co-Enzyme Q-10 is formed into a second single oral unit.

23) A method according to claim 22, wherein a number of first single oral units are placed in a first container, and a number of the second single oral units are placed in a second container.

24) The method of claim 23, wherein the first and second containers are packaged together.

25) The method of claim 22, wherein the first and second single oral units are a tablet.

26) The method of claim 22, wherein the first and second single oral units are a capsule.

27) The method of claim 22, wherein the second single oral unit is a softgelatin capsule.

28) The method of claim 13, wherein the HMG CoA reductase inhibitor component is administered orally to the patient in one daily dose of at least 5 milligrams per day.

29) The method of claim 13, wherein the Co-Enzyme Q-10 is administered orally to the patient in one daily dose containing at least 5 milligrams of Co-Enzyme Q-10.

30) The method of claim 22, wherein binders and carriers are used in the first and second oral units to facilitate the administration of the HMG CoA reductase inhibitor component and the Co-Enzyme Q-10.

31) A process for treating hypercholesterolemia, mixed dyslipidemia, and coronary artery disease comprising the steps of
a) providing a comprising:
i) an IMG CoA reductase inhibitor; and
ii) a CoEnzyme Q-10, and
b) introducing said composition into a human body that is afflicted with a hypercholesterolemia, mixed dyslipidemia, or coronary artery disease.
